(19) United States
(12) Patent Application Publication
(10) Pub. No.: US 2015/0119810 A1
(43) Pub. Date: Apr. 30, 2015

(54) Dosage System

(71) Applicant: Raumedic AG, Munchberg (DE)

(72) Inventors: Thomas Jakob, Selb (DE); Frank Skaper, Leupoldsgrun (DE); Gero Eichelkraut, Dresden (DE); Thomas Braun, Hof (DE); Bernd Spindler, Helmbrechts (DE)

(21) Appl. No.: 14/529,571

(22) Filed: Oct. 31, 2014

(30) Foreign Application Priority Data
Oct. 31, 2013 (DE) 10 2013 222 187.8

(51) Int. Cl. A61M 5/315

(52) U.S. Cl. CPC A61M 5/31563; A61M 5/31555; A61M 2005/3126
USPC 604/189; 604/208

(57) ABSTRACT

The invention relates to a dosage system for setting a dosage volume of a flowable medium. The dosage system comprises a longitudinal axis, a dosage container for receiving the medium, and a dosage piston body which is adapted for sealed displacement in the dosage container between an initial position and a dosage position. In the initial position, the dosage piston body is fully inserted in the dosage container while in the dosage position, the dosage piston body has been pulled out of the dosage container along a path corresponding to the desired dosage amount of the medium. The dosage system further exhibits a draw-up limiting member for limiting a draw-up path of the dosage piston body in the dosage position, the draw-up limiting member being axially securable to the dosage container in one of several limiting positions relative to the longitudinal axis. The dosage system further exhibits a stop for abutment against the draw-up limiting member in order to limit the draw-up path of the dosage piston body in the dosage position. The stop is rigidly axially connected with the dosage piston body in the axial direction.

### CROSS-REFERENCE TO RELATED APPLICATIONS

This application claims the priority of Patent Application Serial No. DE 10 2013 222 187.8 filed on 31 Oct. 2013, pursuant to 35 U.S.C. 119(a)(d), the content of which is incorporated herein by reference in its entirety as if fully set forth herein.

### FIELD OF THE INVENTION

The invention relates to a dosage system for setting a dosage volume of a flowable medium such as a liquid drug.

### BACKGROUND ART

From prior art, dosage systems are already known through prior public use the dosage volumes of which are settable. A frequent drawback of the known dosage systems is that the operation thereof is not user-friendly. Furthermore, many of the known dosage system are not intuitively usable. Also, they generally allow only a few dosage volumes to be set, with the actual dosage volume often greatly deviating from the desired dosage volume. Furthermore, the known dosage systems are difficult to produce and assemble.

### SUMMARY OF THE INVENTION

An object of the invention is therefore to provide a dosage system for setting a dosage volume of a flowable medium which is extremely user-friendly and intuitive. Another object is to provide a dosage system which allows a greatest possible number of dosage volumes to be set in an extremely precise and individually adjustable manner.

This object is achieved according to the invention by a dosage system for setting a dosage volume of a flowable medium, the dosage system comprising a longitudinal axis, a one-piece dosage container for receiving the medium, the dosage container exhibiting at least one communication orifice for communication with surroundings; a dosage piston body extending along the longitudinal axis, the dosage piston body being adapted for sealed displacement in the dosage container between an initial position in which the dosage piston body is fully inserted in the dosage container, and a dosage position in which the dosage piston body has been pulled out of the dosage container along a path corresponding to the desired dosage amount of the medium; a draw-up limiting member for limiting a draw-up path of the dosage piston body in the dosage position, the draw-up limiting member being axially securable to the dosage container in one of several limiting positions relative to the longitudinal axis, the draw-up limiting member being displaceable relative to the dosage container and radially to the longitudinal axis between an inner limiting position axially secured to the dosage container and an outer position; and a stop for abutment against the draw-up limiting member in order to limit the draw-up path of the dosage piston body in the dosage position, the stop being rigidly connected to the dosage piston body in the axial direction, the draw-up limiting member being configured in such a way that it is contactable with the stop only if it is in its radially inner position.

The gist of the invention is that a draw-up limiting member axially secured to the dosage container in a limiting position allows the draw-up path of the dosage piston body to be limited. To this end, the dosage piston body is provided with a corresponding, axially fixed stop. The maximum dosage volume is settable on the dosage container depending on the axial position of the draw-up limiting member.

The draw-up limiting member forms a counter-stop for the stop. In contrast to a purely visual setting, said tactile, in other words mechanical, stop prevents administration of wrong doses. In conventional measuring syringes, the rate of wrong dosages amounts to an average of approx. 35% relative to the dosage volume while in conventional measuring spoons, the rate of wrong dosages even amounts to an average of approx. 85% relative to the dosage volume. Consequently, the tactile or mechanical stop results in an increased safety, which is particularly advantageous when it comes to highly active drugs and low dosage volumes, enabling even visually impaired people to set or provide an exact dosage.

The draw-up limiting member is radially displaceable. It is advantageous if the draw-up limiting member is displaceable perpendicularly to the longitudinal axis between its radial positions. An oblique displacement relative to the longitudinal axis is alternatively conceivable.

The draw-up limiting member is only able to abut against, in other words interact with the stop if said draw-up limiting member is in its radially inner position. Conversely, the draw-up limiting member is inactive in its radially outer position, in other words it is unable to interact with the stop.

The dosage piston body is preferably freely displaceable between the draw-up limiting member and the stop until it comes to rest against the stop. Ideally, the limiting positions are defined by first positive fit means at the dosage container. It is advantageous if at least a second positive fit means is arranged at the draw-up limiting member for interaction with at least one of the first positive fit means. Alternatively, the draw-up limiting member is axially securable to the dosage container in an infinitely variable manner, in other words there are no precise limiting positions. It is advantageous if the connection between at least one first positive fit means at the dosage container and at least one second positive fit means at the draw-up limiting member is a plug-in connection or a snap-in locking connection.

Advantageously, the dosage system is used in pediatrics or in the treatment of elderly patients.

The at least one communication orifice allows the medium to enter the dosage container. The medium disposed in the receiving space is further dispensable from the receiving space via the at least one communication orifice.

When the dosage piston body is in the pulled-out position, the dosage amount has a maximum volume while it has a minimum volume in the pushed-in position of the dosage piston body.

The draw-up limiting member is preferably configured in the manner of a bracket. Conveniently, the draw-up limiting member is symmetric, more preferably U-shaped.

Ideally, the dosage system is configured in the manner of a syringe. The design is preferably similar to that of known measuring syringes users are familiar with. The dosage system is preferably extremely compact.

It is advantageous if the dosage system does not have any loose parts, ensuring that the parts of the dosage system cannot be lost.

Ideally, the draw-up limiting member has a different colour than the dosage container which ensures a particularly fast and secure visual perception thereof.

It is advantageous if the dosage container is made of a plastic material, preferably of a transparent plastic material. Advantageously, the dosage piston body is made of a plastic material. Conveniently, the draw-up limiting member is made of a plastic material as well.

According to one embodiment, the draw-up limiting member is axially displaceable in the direction of the longitudinal axis between at least two dosage positions on the outside of the dosage container if said draw-up limiting member is in its radially outer position. More preferably, the draw-up limiting member is axially securable to the outside of the dosage container in at least five, more preferably in at least ten, more preferably in at least 15, different positions which are spaced from each other in the direction of the longitudinal axis. Preferably the positions are predetermined positions. Alternatively, the draw-up limiting member is displaceable in an infinitely variable manner in the direction of the longitudinal axis, in other words it is axially securable to the outside of the dosage container in an infinitely variable manner. Consequently, the dosage positions may be predetermined or non-predetermined, in other words freely selectable. This embodiment provides an extremely fine measurement scale with a large number of measuring increments. Ideally, an external adjustment device is provided as well.

The draw-up limiting member is reversibly or irreversibly axially displaceable in the direction of the longitudinal axis on the outside of the dosage container between at least two dosage positions when in the radially outer position.

It is advantageous if it is possible to pull out the piston body in a radially outer cleaning position of the draw-up limiting member.

When moved axially relative to the dosage container, the draw-up limiting member disposed in the radially outer position provides a feedback, preferably an acoustic and/or tactile feedback, with reference to the limiting positions, thus allowing the limiting position to be identified particularly easily. Ideally, there is an acoustic and/or tactile feedback when the first and second positive fit means are interacting during a relative movement between the draw-up limiting member and the dosage container. Advantageously, the draw-up limiting member makes a clattering sound when moving along the dosage container during a relative movement between the dosage container and the draw-up limiting means. It is advantageous if the first positive fit means are positive fit receptacles, orifices or windows which are arranged at the dosage container at an axial distance from each other in the direction of the longitudinal axis.

In one embodiment, a retaining device is active between the draw-up limiting member and the dosage container, the retaining device limiting the radial displacement of the draw-up limiting member in the outward direction or making it harder to do so, which results in a loss-proof arrangement of the draw-up limiting member at the dosage container. Alternatively, the draw-up limiting member is detachable from the dosage container. This is in particular advantageous for cleaning purposes. The retaining device hinders, preferably prevents, a radial movement of the draw-up limiting member from the radially inner position towards the radially outer position and/or from the radially outer position to a position that lies beyond said radially outer position.

A snap-in locking connection in the desired dosage position between the draw-up limiting member and the dosage container is extremely safe and easily producible. Alternatively, a plug-in connection is provided.

In one embodiment, the draw-up limiting member encompasses an outer circumferential portion of the dosage container. Advantageously, this applies both to the radially inner and to the radially outer position of the draw-up limiting member.

The embodiment in which the draw-up limiting member exhibits a marking which, together with a measurement scale provided at the dosage container, indicates a dosage amount of the medium to be set allows the settable dosage amount to be adjusted in an extremely precise and simple manner by means of the draw-up limiting member.

The embodiment in which the stop is arranged adjacent to a piston bottom of the dosage piston body in order to discharge the medium allows a maximum dosage amount to be set that is extremely large.

The one-piece configuration between the stop and the dosage piston body results in a dosage system which is extremely operationally safe and inexpensive to produce.

The embodiment in which the stop is rigidly connected with a dosage piston arm extending parallel to the dosage piston body, the dosage piston arm being rigidly axially connected with a base body of the dosage piston body, results in a dosage system which is extremely operationally safe and particularly easy to produce.

The embodiment in which the dosage piston arm is displaceably guided in a guide seat of the dosage container results in a protected arrangement of the dosage piston arm. Furthermore, the movement thereof is particularly well predeterminable.

The embodiment in which the dosage container exhibits a receiving space for receiving the medium, the receiving space being arranged spatially separately from the guide seat, results in that the receiving space is not reduced or affected by the guide seat. Consequently, the setting of the dosage volume is not separate from the dosage of the medium.

Two preferred embodiments will hereinafter be described by way of example with reference to the attached drawing.

### BRIEF DESCRIPTION OF THE DRAWING

FIG. 1 shows a perspective view of a dosage system according to the invention, the draw-up limiting member being in its radially inner limiting position;

FIG. 2 shows a side view of the dosage system shown in FIG. 1;

FIG. 3 shows a cross-sectional view of the dosage system along section line III-III shown in FIG. 2;

FIG. 4 shows a longitudinal sectional view of the dosage system shown in FIGS. 1 and 2;

FIG. 5 shows a longitudinal sectional view corresponding to FIG. 4, the draw-up limiting member being in an axially different dosage position;

FIG. 6 shows a side view corresponding to FIG. 2, the draw-up limiting member being in its radially outer position;

FIG. 7 shows a cross-sectional view corresponding to section line VII-VII illustrated in FIG. 6;

FIG. 8 shows a longitudinal sectional view of the dosage system shown in FIG. 6;

FIG. 9 shows a cross-sectional view corresponding to FIG. 3 of an inventive dosage system according to a second embodiment, the draw-up limiting member being in its radially inner limiting position; and

FIG. 10 shows a cross-sectional view corresponding to FIG. 6 of the dosage system shown in FIG. 9, the draw-up limiting member being in its radially outer position.

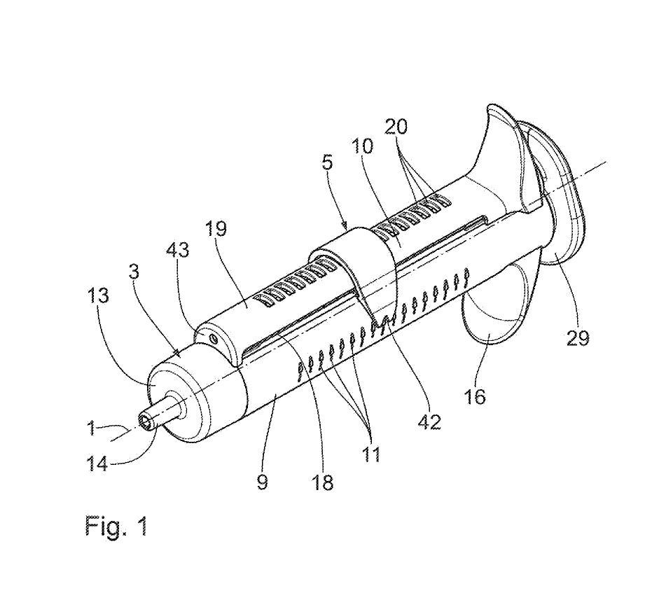

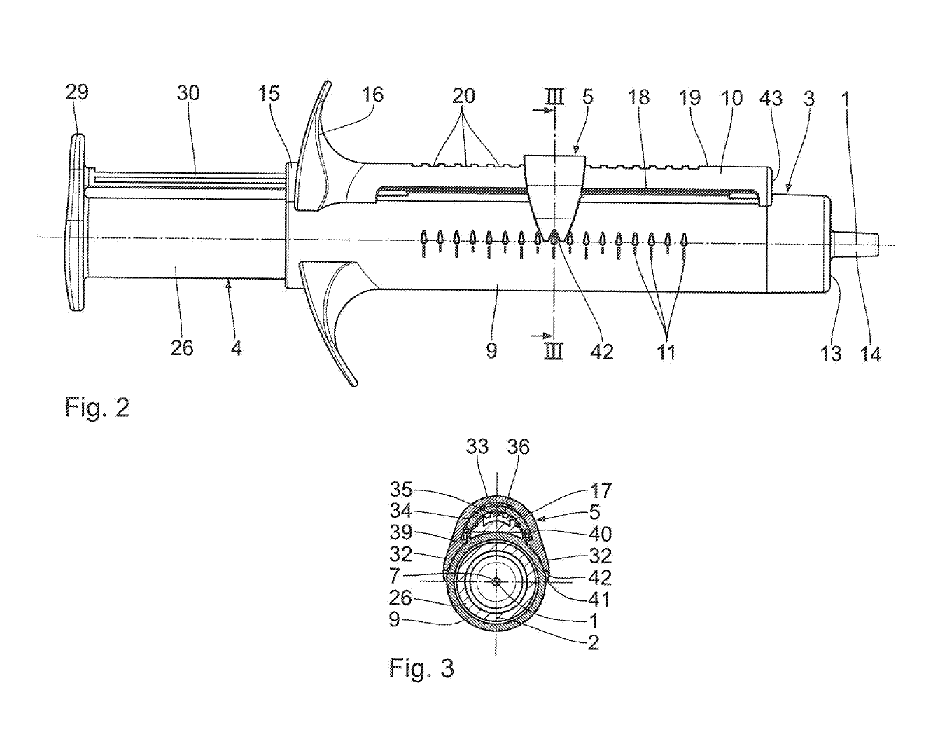

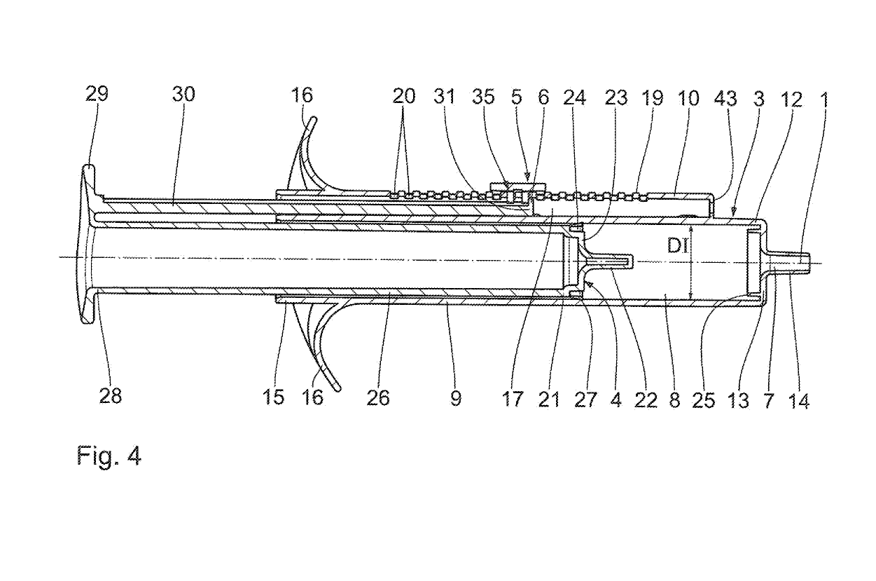

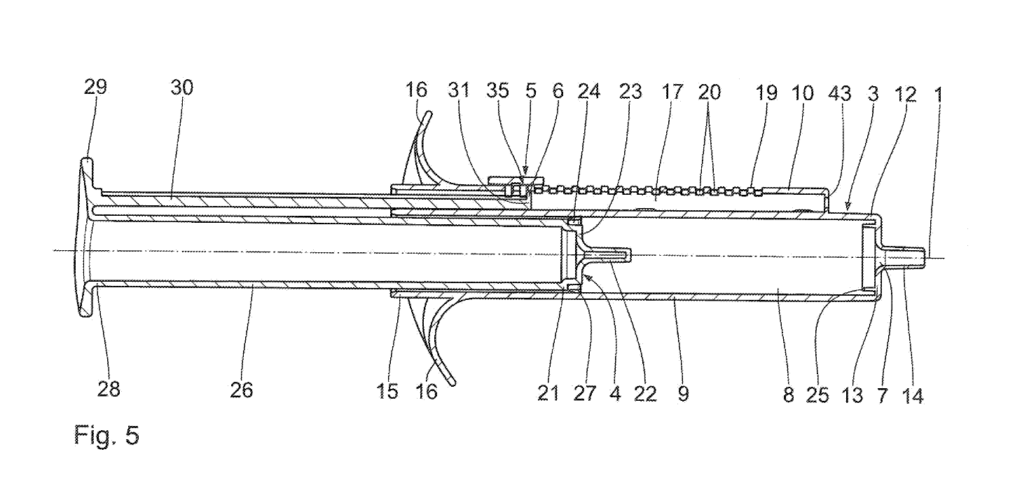

### DESCRIPTION OF THE PREFERRED EMBODIMENTS

A dosage system shown in the drawing has a longitudinal axis 1 and is symmetric relative to a symmetry plane 2. The longitudinal axis 1 is in the symmetry plane 2.

The dosage system comprises a dosage container 3 and a dosage piston body 4 which is displaceable in the dosage container 3 relative thereto in the direction of the longitudinal axis 1. The dosage container 3 serves as a receptacle for a flowable medium. The dosage container 3 defines a storage space 8 for the flowable medium (not shown) and has a communication orifice 7 for communication with the surroundings. The dosage piston body 4 is axially displaceable in the dosage container 3 relative to the dosage container 3 between an initial position and a dosage position. In the initial position, the dosage piston body 4 is fully inserted into the dosage container 3 while in the dosage position, it has been pulled out of the dosage container 3 along a path corresponding to the desired dosage amount of the medium. The expressions “axial” and “radial” used throughout this disclosure each refer to the longitudinal axis 1.

The dosage system further exhibits a draw-up limiting member 5 which is axially securable to the dosage container 3 in various limiting positions spaced from each other in the direction of the longitudinal axis 1 so as to spatially limit the maximum draw-up path of the dosage piston body 4. The dosage piston body 4 is provided with a stop 6 for mechanical interaction with the draw-up limiting member 5 in the dosage position. Depending on the axial arrangement of the draw-up limiting member 5 relative to the dosage container 3, the maximum draw-up path of the dosage piston body 4, and therefore the maximum dosage amount, is individually adjustable. The maximum draw-up path is defined by the draw-up limiting member 5.

When the dosage piston body 4 is pulled from its initial position to axially move to its dosage position, a liquid medium may be drawn into the receiving space 8 via the communication orifice 7. If, however, the dosage piston body 4 is pushed from a dosage position to axially move to its initial position, the medium disposed in the receiving space 8 may be dispensed, in other words ejected, via the communication orifice 7.

The dosage container 3 comprises a measuring sleeve 9 extending along the longitudinal axis 1 and a setting projection 10 laterally attached to the measuring sleeve 9. The measuring sleeve 9 and the setting projection 10 are formed in one piece, more preferably injection-moulded, of a plastic material.

The measuring sleeve 9 has a measurement scale 11 extending along the longitudinal axis 1 and is configured as a cylindrical body which has a substantially constant internal diameter DI in the direction of the longitudinal axis 1 and is closed at a first end 12 by means of a measuring sleeve bottom 13. The measuring sleeve bottom 13 extends perpendicularly to the longitudinal axis 1. The communication orifice 7 is arranged centrally in the measuring sleeve bottom 13. A tube section 14 the internal diameter of which is preferably considerably smaller than the internal diameter DI of the measuring sleeve 9 adjoins the outside of the measuring sleeve bottom 13 in the region of the communication orifice 7. The measuring sleeve 9 and the measuring sleeve bottom 13 together define the receiving space 8. Alternatively, the tube section 14 is omitted so that the measuring sleeve 9 has a substantially constant diameter along its entire length in order to dispense the flowable medium. Alternatively, a Luer lock connector is provided instead of the tube section 14 allowing for instance other syringes, infusion instruments or the like to be connected thereto.

The measuring sleeve 9 further has a second end 15 which is arranged opposite to the first end 12. In the region of the second end 15, two handle pieces 16 protrude in the radially outward direction. The two handle pieces 16 are arranged opposite to each other. A different arrangement or configuration of handle pieces 16 is alternatively conceivable.

The setting projection 10 extends along the longitudinal axis 1 at the side of the measuring sleeve 9. It substantially starts at the second end 15 and ends adjacent to but spaced from the first end 12. Alternatively, it extends up to the first end 12. The setting projection 10 adjoins the side of the measuring sleeve 9 in a circumferential region of approximately 80° about the central longitudinal axis 1.

The setting projection 10 is hollow, thus providing an outward boundary to an inner guide seat. The setting projection 10 has an arc-shaped cross-section. Other cross-sectional shapes are alternatively conceivable. The setting projection 10 is axially open at the second end 15 while it is substantially axially closed at its other end 43 opposite thereto. Alternatively, the setting projection 10 is at least partly open at the end 43.

Between the measuring sleeve 9 and the setting projection 10, two lateral retaining guide grooves 18 extend along the longitudinal axis 1 of the dosage container 3 which are also parallel to each other, are arranged opposite to each other and are laterally outwardly opening in opposite directions. According to an alternative embodiment, the dosage container 3 is provided with more than two lateral retaining guide grooves 18.

The outside 19 of the setting projection 10 remote from the measuring sleeve 9 is provided with first positive fit means 20 which are spaced from each other in a row extending along the longitudinal axis 1. The first positive fit means 20 are each configured as orifices or windows. They are open in the direction of the guide seat 17. Preferably, the first positive fit means 20 have a rectangular cross-section and are parallel to each other. Other cross-sectional shapes are alternatively conceivable. Alternatively, one single first positive fit means is provided instead of several positive fit means, which single first positive fit means extends at the measuring sleeve 9 along the longitudinal axis 1 and is therefore continuous along the longitudinal axis 1, thus allowing the draw-up path of the dosage piston body 4 to be limited in an infinitely variable manner.

At its first end 21, the dosage piston body 4 has a displacement body or ejection body 22 protruding in the direction of the longitudinal axis 1, the external diameter thereof substantially corresponding to the internal diameter of the tube section 14. The axial length of the displacement body 22 substantially corresponds to the axial length of the tube section 14. It is preferably slightly larger. The displacement body 22 is substantially cylindrical and has a ring-shaped cross-section. It is closed at the front. A fully cylindrical configuration is alternatively conceivable.

The displacement body 22 is adjoined by a radially protruding piston bottom 23 which is a component of the dosage piston body 4. The piston bottom 23 has an external diameter which is smaller than the internal diameter DI of the measuring sleeve 9 so that an annular gap 24 running around the longitudinal axis 1 is provided in the region of the piston bottom 23. The annular gap 24 is radially outwardly bounded by a ring-shaped sealing protrusion 27 which sealingly abuts against the inside of the wall of the measuring sleeve 9 and is flexible to a certain extent.

An annular web 25 protrudes from the measuring sleeve bottom 13 in the direction of the longitudinal axis 1, the annular web 25 running around the longitudinal axis 1 at a distance from the wall of the measuring sleeve 9. When the dosage piston body 4 has been fully inserted into the measuring sleeve 9, the annular web 25 positively engages the annular gap 24; as a result, residual amounts can be discharged from the dosage system particularly effectively, in other words completely.

The piston bottom 23 is adjoined by a base body 26 of the dosage piston body 4 in such a way that the annular gap 24 is formed, the base body 26 being configured cylindrically. The base body 26 has an external diameter which substantially corresponds to the internal diameter DI of the measuring sleeve 9. The external diameter of the base body 26 is preferably slightly smaller than the internal diameter DI. The annular web 25 starts at the base body 26.

Opposite to the first end 21, the dosage piston body 4 has a second end 28 where an actuation protrusion 29 is disposed which protrudes outwardly to the side.

At a distance from the base body 26, a dosage piston arm 30 protrudes from the actuation protrusion 29, the dosage piston arm 30 extending parallel to the longitudinal axis 1 in the direction of the end 43 of the setting projection 10. The dosage piston body 30 has an axial length which substantially corresponds to the axial length of the setting projection 10. The radial distance between the base body 26 and the dosage piston arm 30 corresponds approximately to the thickness of the wall of the measuring sleeve 9.

The dosage piston arm 30 has a free axial end 31 at which the stop 6 is arranged. The stop 6 protrudes radially outwardly relative to the dosage piston arm 30. It is axially immovable relative to the base body 26.

Via the second end 15, the dosage piston arm 30 engages the guide seat 17 in which it is displaceably guided. The coupling with the actuation protrusion 29 ensures that when the base body 26 is displaced, the dosage piston arm 30, which carries the stop 6, is correspondingly displaced in the same direction.

The draw-up limiting member 5 is configured in the manner of a bracket and has two opposite U-legs 32 which are interconnected via an outer U-bottom 33. The U-legs 32 are resiliently deflectable relative to each other to a certain extent.

The U-bottom 33 has an inner side 34 facing the setting projection 10, the curvature of the inner side 34 being substantially adapted to the outer curvature of the setting projection 10. Two second positive fit means 35 protrude from the inner side 34 of the U-bottom 33 in the direction of the longitudinal axis 1. The second positive fit means 35 are axially spaced from each other in the direction of the longitudinal axis 1. The second positive fit means 35 are arranged and configured in such a way that they are snap-lockable with two first positive fit means 20 arranged adjacent to each other, which ensures that the draw-up limiting member 5 is secured relative to the setting projection 10 and therefore relative to the dosage container 3. Each second positive fit means 35 comprises two second positive fit protrusions 36. Each second positive fit protrusion 36 has a laterally outwardly protruding snap-in lug 37 which in turn has a retaining surface 38 facing the inner side 34 of the U-bottom 33. Alternatively, exactly one second positive fit means 35 is provided.

A retaining lug 39 protrudes inwardly from each U-leg 32, the retaining lug 39 having a second retaining surface 40 facing the inner side 34 of the U-bottom 33. The two retaining lugs 39 protrude from the U-legs 32 towards each other, with each of them being arranged between the U-bottom 33 and free ends 41 of the U-legs 32. Each U-leg 32 has a respective curvature between its retaining lug 39 and its free end 41, the curvature being adapted to the outer curvature of the measuring sleeve 9.

In the region of the free ends 41, each U-leg 32 is provided with a marking 42 which is configured as a V-shaped recess in this embodiment. Other configurations of the marking 42 are alternatively conceivable.

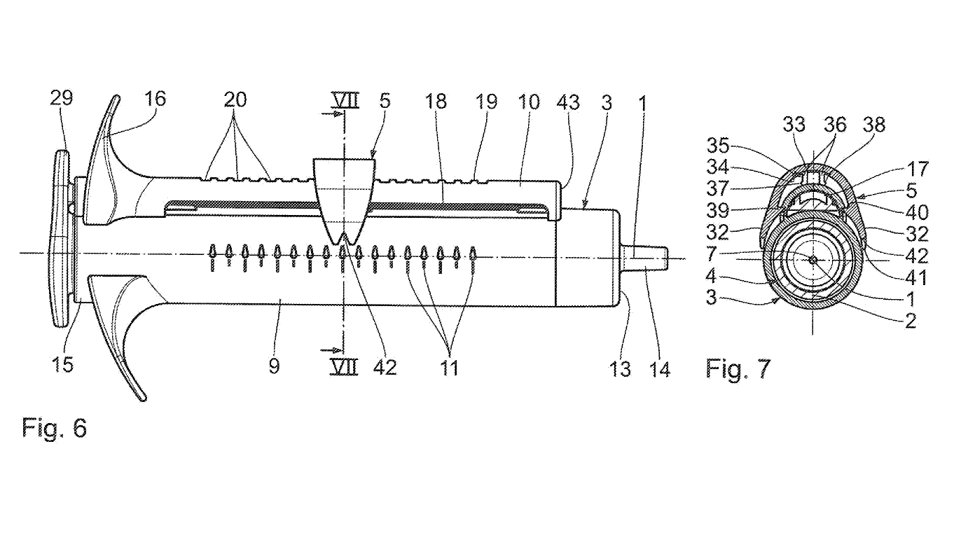

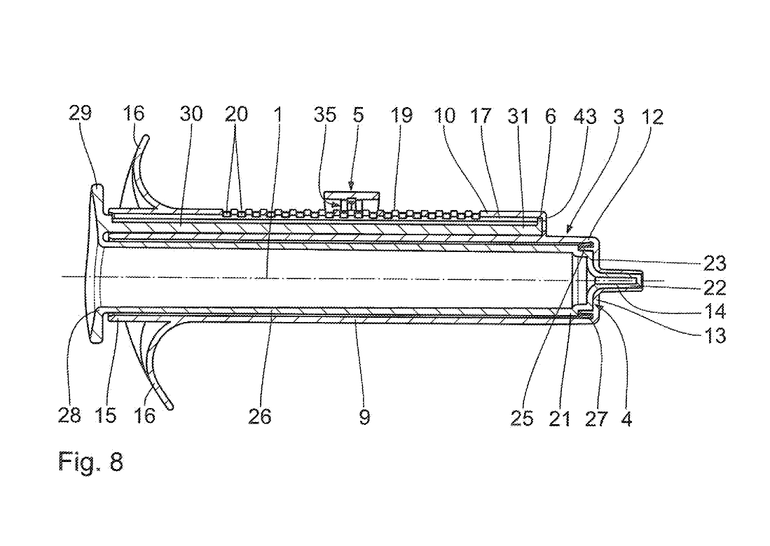

The following is a more detailed description of the dosage system when in use. This description starts from a condition in which the draw-up limiting member 5 is in its radially outer, inactive position. This condition is illustrated in FIGS. 6 to 8.

In this condition, the second positive fit means 35 are disengaged from the first positive fit means 20 in such a way that the draw-up limiting member 5 is displaceable along the longitudinal axis 1 relative to the measuring sleeve 9, in other words the dosage container 3. Axial displacement of the draw-up limiting member 5 relative to the measuring sleeve 9 is guided by the retaining lugs 39 which engage the retaining guide grooves 18 laterally from outside. The engagement of the retaining lugs 39 with the guide grooves 18 prevents the draw-up limiting member 5 from being removed in the radially outward direction or makes it harder to do so.

The retaining surfaces 40 engaging with the setting projection 10 in the region of the retaining guide grooves 18 prevent the draw-up limiting member 5 from being removed from the dosage container 3 in the radial direction or make it harder to do so.

When the draw-up limiting member 5 is being displaced, the second positive fit means 35 slightly engage the first positive fit means 20 for a short time, which results in an acoustic feedback in the form of a clattering sound.

The draw-up limiting member 5 is then axially secured to the setting projection 10 in a limiting position. The limiting position then defines a desired dosage amount of the medium. Setting of the desired dosage amount is facilitated by the measurement scale 11 and the marking 42 provided at the dosage container 3. The marking 42 indicates the respective maximum dosage amount that is receivable by the receiving space 8 due to the setting of the draw-up limiting member 5.

The draw-up limiting member 5 is axially secured relative to the dosage container 3 by exerting a manual force on the U-bottom 33 from outside in the direction of the longitudinal axis 1 in such a way that the U-bottom 33 moves closer to the outer side 19 of the setting projection 10, with the second positive fit means 35 engaging the corresponding first positive fit means 20 adjacent thereto. The radially inner position of the draw-up limiting member 5 is reached when the inner side 34 of the U-bottom 33 is at least partly in contact with the outer side 19 of the setting projection 10 or is arranged directly adjacent thereto so that the second positive fit means 35 are in a locking engagement with the corresponding first positive fit means 20 adjacent thereto.

The second positive fit means 35 engage the guide seat 17 from outside via the first positive fit means 20 and protrude inwardly relative to the first positive fit means 20. The first retaining surfaces 38 reach behind the wall of the setting projection 10 bounding the corresponding first positive fit means 20, thus preventing the draw-up limiting member 5 from being moved back into its radially outer position or making it harder to do so.

In the region between the free ends 41 and the retaining lugs 39, the U-legs 32 are in a surface-to-surface contact with the outside of the measuring sleeve 9. The retaining lugs 39 are now remote from the retaining guide grooves 18.

The draw-up limiting member 5 is now secured to the dosage container 5 both axially and radially and in the circumferential direction about the longitudinal axis 1. An external snap-in locking engagement is provided.

The second positive fit means 35 facing the axial end 43 of the setting projection 10 protrudes into the guide seat 17 so as to form a counter stop for the stop 6 which is disposed in the guide seat 17 as well. As a result, the draw-up path of the dosage piston body 4 is limited. In the dosage position, the stop 6 abuts against the side of the counter stop facing the end 43 of the setting projection 10, thus preventing the dosage piston body 4 from being pulled out any further.

The snap-in lugs 37 and the retaining lugs form part of a retaining device. Axial displacement of the dosage piston body 4 is facilitated by using the handle pieces 16 and the actuation protrusion 29 which allow corresponding displacement forces to be exerted.

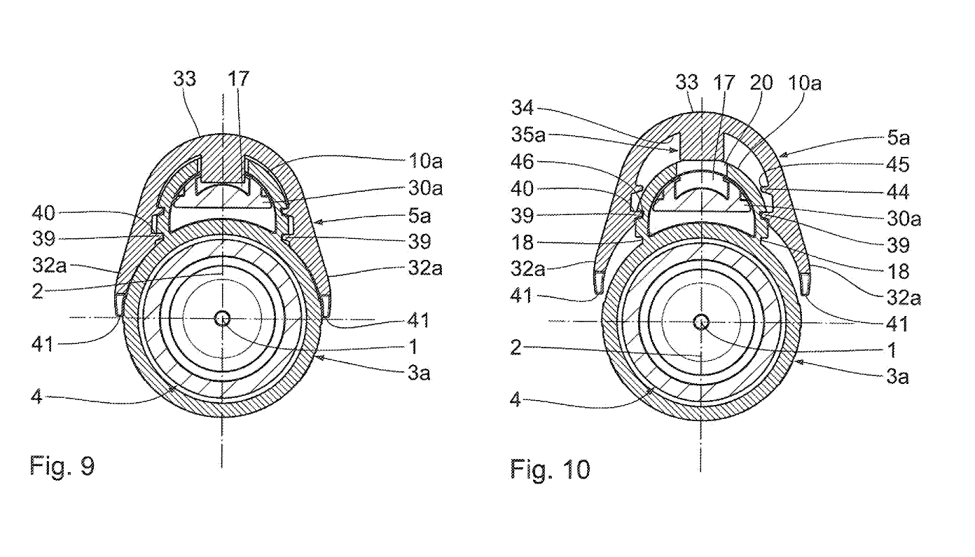

The following is a description of a second embodiment with reference to FIGS. 9 and 10. Constructionally identical parts are designated by the same reference numerals as in the preceding embodiment to which reference is made. Constructionally different parts with the same function are designated by the same reference numerals as in the preceding embodiment followed by an “a”.

In this embodiment, the dosage container 3a and the draw-up limiting member 5a differ from those of the preceding embodiment in terms of their constructional design.

In contrast to the preceding embodiment, the draw-up limiting member 5 is additionally provided with two second retaining lugs 44 in addition to the retaining lugs 39. One of the second retaining lugs 44 protrudes inwardly from each U-leg 32a, said retaining lug 44 exhibiting a retaining surface 45 facing the inner side 34 of the U-bottom 33. The two second retaining lugs 44 protrude from the U-legs 32a towards each other and are in each case arranged between the U-bottom 33 and the retaining lugs 39, said retaining lugs 39 thus forming first retaining lugs 39. The second retaining lugs 44 are arranged adjacent to the first retaining lugs 39 and are parallel thereto. They are arranged more closely to the first retaining lugs 39 than to the U-bottom 33. The second retaining lugs 44 have a shape that is substantially identical to that of the first retaining lugs 39.

A second positive fit means 35a protrudes from the inner side 34 of the U-bottom 33 in the direction of the longitudinal axis 1. In contrast to the preceding embodiment, the second positive fit means 35a is configured as a plug-in protrusion in this embodiment. The second positive fit means 35a is configured in the manner of a plate or block. It is not provided with any snap-in lugs or the like. Alternatively, more than exactly one second positive fit means 35a is provided.

Compared to the preceding embodiment, two lateral second retaining guide grooves 46 extend at the setting projection 10a of the dosage container 3a along the longitudinal axis 1, said second retaining guide grooves 46 also extending parallel to each other and to the retaining guide grooves 18. The retaining guide grooves 18 form first retaining guide grooves 18. The second retaining guide grooves 46 are arranged opposite to each other. They are laterally outwardly open in opposite directions. The second retaining guide grooves 46 are adjacent to the first retaining guide grooves 18. The distance between a first retaining guide groove 18 and the second retaining guide groove 46 arranged adjacent thereto substantially corresponds to the distance of the first retaining lug 39 from the second retaining lug 44 arranged adjacent thereto.

The following is a description of the dosage system according to the second embodiment when in use. Said description starts from a condition in which the draw-up limiting member 5a is in its radially outer, inactive position. This condition is illustrated in FIG. 10.

In this condition, the second positive fit means 35a is disengaged from the first positive fit means 20 in such a way that the draw-up limiting member 5a is displaceable along the longitudinal axis 1 relative to the dosage container 3a. Axial displacement of the draw-up limiting member 5a relative to the dosage container 3a is guided by the first retaining lugs 39 which engage the second retaining guide grooves 46 laterally from outside. Engaging the setting projection 10a in the region of the second retaining guide grooves 46, the retaining surfaces 40 facing said setting projection 10a prevent the draw-up limiting member 5a from being removed from the dosage container 3a in the radially outward direction or make it harder to do so. The second retaining lugs 44 have substantially no guide or retaining function in this embodiment.

In a region between the free ends 41 and the first retaining lugs 39, the U-legs 32a run substantially at a distance from the dosage container 3a.

When the draw-up limiting member 5a is displaced axially to adjust the dosage volume, the second positive fit means 35a slightly engages the first positive fit means 20 for a short time, which produces an acoustic feedback in the form of a clattering sound.

The draw-up limiting member 5a is then axially secured to the setting projection 10a in a desired limiting position. The draw-up limiting member 5a is axially secured relative to the dosage container 3a by exerting a manual force on the U-bottom 33 from outside in the direction of the longitudinal axis 1 in such a way that the U-bottom 33 moves closer to the outer side 19 of the setting projection 10a. The radially inner position of the draw-up limiting member 5a is reached when the inner side 34 of the U-bottom 33 is at least partly in contact with the outer side 19 of the setting projection 10a or is arranged directly adjacent thereto so that the second positive fit means 35a is in a releasable plug-in connection with the corresponding first positive fit means 20 adjacent thereto.

In the radially inner position of the draw-up limiting member 5a, the second positive fit means 35a engages the guide seat 17 from outside via the adjacent first positive fit means 20 and protrudes inwardly relative to the first positive fit means 20, thus forming a counter stop for the stop 6. The positive fit connection between the positive fit means 20, 35 ensures that the draw-up limiting member 5a is axially secured to the dosage container 3a.

When the draw-up limiting member 5a is moved from the radially outer to the radially inner position, the first retaining lugs 39 are disengaged from the second retaining guide grooves 46.

In the radially inner position of the draw-up limiting member 5a, the first retaining lugs 39 are in a snap-in locking engagement with the first retaining guide grooves 18 while the second retaining lugs 44 are in a snap-in locking engagement with the second retaining guide grooves 46. The retaining surfaces 40 and 46 of the first and second retaining lugs 39, 44, respectively, facing the U-bottom 33 and abutting against the setting projection 10a prevent the draw-up limiting member 5a from being moved in the radially outward direction or make it harder to do so. It is advantageous if the function of the first retaining lugs 39 is to radially secure the draw-up limiting member 5a.

The draw-up limiting member 5a is now secured to the dosage container 3a both axially and radially and in the circumferential direction about the longitudinal axis 1.

Ideally, the distance between the first retaining guide groove 18 and the second retaining guide groove 46 is smaller than the distance between the second retaining guide groove 46 and the first positive fit means 20 or the apex region of the setting projection 10a. This allows the draw-up limiting member 5a to be moved to a radially outer cleaning position particularly easily.

1. A dosage system for setting a dosage volume of a flowable medium, the dosage system comprising
a) a longitudinal axis,
b) a one-piece dosage container for receiving the medium;
i) the dosage container exhibiting at least one communication orifice for communication with surroundings;
c) a dosage piston body extending along the longitudinal axis, the dosage piston body being adapted for sealed displacement in the dosage container between
i) an initial position in which the dosage piston body is fully inserted in the dosage container; and
ii) a dosage position in which the dosage piston body has been pulled out of the dosage container along a path corresponding to the desired dosage amount of the medium;
d) a draw-up limiting member for limiting a draw-up path of the dosage piston body in the dosage position, the draw-up limiting member being axially securable to the dosage container in one of several limiting positions relative to the longitudinal axis,
i) the draw-up limiting member being displaceable relative to the dosage container and radially to the longitudinal axis between an inner limiting position axially secured to the dosage container and an outer position; and
e) a stop for abutment against the draw-up limiting member in order to limit the draw-up path of the dosage piston body in the dosage position,
i) the stop being rigidly connected to the dosage piston body in the axial direction,
ii) the draw-up limiting member being configured in such a way that it is contactable with the stop only if it is in its radially inner position.

2. The dosage system according to claim 1, wherein the draw-up limiting member is axially displaceable in the direction of the longitudinal axis between at least two dosage positions on the outside of the dosage container if the draw-up limiting member is in its radially outer position.

3. The dosage system according to claim 1, wherein when moved axially relative to the dosage container, the draw-up limiting member disposed in the radially outer position provides a feedback with reference to the limiting positions.

4. The dosage system according to claim 3, wherein the feedback provided by the draw-up limiting member is at least one of the group comprising an acoustic and a tactile feedback.

5. The dosage system according to claim 1, comprising a retaining device acting between the draw-up limiting member and the dosage container, the retaining device limiting the radial displacement of the draw-up limiting member in the outward direction.

6. The dosage system according to claim 1, wherein the draw-up limiting member is snap-in lockable with the dosage container in the desired dosage position by means of a snap-in locking connection.

7. The dosage system according to claim 1, wherein the draw-up limiting member encompasses an outer circumferential portion of the dosage container.

8. The dosage system according to claim 1, wherein the draw-up limiting member exhibits a marking which, together with a measurement scale provided at the dosage container, indicates a dosage amount of the medium to be set.

9. The dosage system according to claim 1, wherein the stop is arranged adjacent to a piston bottom of the dosage piston body in order to discharge the medium.

10. The dosage system according to claim 1, wherein the stop is formed in one piece with the dosage piston body.

11. The dosage system according to claim 1, wherein the stop is rigidly connected with a dosage piston arm extending parallel to the dosage piston body, the dosage piston arm being rigidly axially connected with a base body of the dosage piston body.

12. The dosage system according to claim 11, wherein the dosage piston arm is displaceably guided in a guide seat of the dosage container.

13. The dosage system according to claim 12, wherein the dosage piston body exhibits an axial end region, the dosage piston arm starting at the axial end region so as to extend adjacent to the base body of the dosage piston body.

14. The dosage system according to claim 12, wherein the dosage container exhibits a receiving space for receiving the medium, the receiving space being arranged spatially separately from the guide seat.

15. The dosage system according to claim 1, wherein when disposed in the radially outer position, the draw-up limiting member is unable to interact with the stop.
